(19) United States
(12) Patent Application Publication
(10) Pub. No.: US 2011/0040309 A1
(43) Pub. Date: Feb. 17, 2011

(54) IMPLANTABLE RESTRICTION DEVICE WITH SPACER

(76) Inventors: Daniel F. Dlugos, JR., Middletown, OH (US); Chad P. Boudreaux, Cincinnati, OH (US); Robert P. Gill, Mason, OH (US); Steven G. Hall, Cincinnati, OH (US); Christopher J. Hess, Cincinnati, OH (US); Jerome R. Morgan, Cincinnati, OH (US); Frederick E. Shelton, IV, New Vienna, OH (US); Jeffrey S. Swayze, Hamilton, OH (US); Omar J. Vakharia, Cincinnati, OH (US); Lauren S. Weaner, Cincinnati, OH (US); William Bruce Weisenburgh, II, Maineville, OH (US); Andrew M. Zwolinski, Cincinnati, OH (US)

Correspondence Address: WELSH FLAXMAN & GITLER LLC, 2000 DUKE STREET, SUITE 100, ALEXANDRIA, VA, 22314, US

(21) Appl. No.: 12/541,579

(22) Filed: Aug. 14, 2009

(51) Int. Cl. A61B 17/00

(52) U.S. Cl. 606/151

(57) ABSTRACT

An implantable restriction device includes a belt and a balloon secured to the belt for engagement with tissue when the implantable restriction device is positioned about the stomach. A spacer is associated with the belt for maintaining the restriction device in a spaced relation with a staple line employed during gastric reduction surgery.

### BACKGROUND OF THE INVENTION

1. Field of the Invention

The invention relates to an implantable restriction device. More particularly, the invention relates to protecting both the implantable restriction device and the internal tissue or lumen, such as, stomach tissue, during application and use of the implantable restriction device by spacing the implantable restriction device from a staple line or other portion of the stomach during application thereof.

2. Description of the Prior Art

Morbid obesity is a serious medical condition. In fact, morbid obesity has become highly pervasive in the United States, as well as other countries, and the trend appears to be heading in a negative direction. Complications associated with morbid obesity include hypertension, diabetes, coronary artery disease, stroke, congestive heart failure, multiple orthopedic problems and pulmonary insufficiency with markedly decreased life expectancy. With this in mind, and as those skilled in the art will certainly appreciate, the monetary and physical costs associated with morbid obesity are substantial. In fact, it is estimated the costs relating to obesity are in excess of one hundred billion dollars in the United States alone.

A variety of surgical procedures have been developed to treat obesity. The most common currently performed procedure is Roux-en-Y gastric bypass (RYGB). This procedure is highly complex and is commonly utilized to treat people exhibiting morbid obesity. Other forms of bariatric surgery include Fobi pouch, bilio-pancreatic diversion, and gastroplastic or “stomach stapling”. In addition, implantable devices are known which limit the passage of food through the stomach and affect satiety.

In view of the highly invasive nature of many of these procedures, efforts have been made to develop less traumatic and less invasive procedures. Gastric-banding is one of these methods. Gastric-banding is a type of gastric reduction surgery attempting to limit food intake by reducing the size of the stomach. In contrast to RYGB and other stomach reduction procedures, gastric-banding does not require the alteration of the anatomy of the digestive tract in the duodenum or jejunum.

Since the early 1980's, gastric bands have provided an effective alternative to gastric bypass and other irreversible surgical weight loss treatments for the morbidly obese. Several alternate procedures are performed under the heading of gastric-banding. Some banding techniques employ a gastric ring, others use a band, some use stomach staples and still other procedures use a combination of rings, bands and staples. Among the procedures most commonly performed are vertical banded gastroplasty (VBG), silastic ring gastroplasty (SRG) and adjustable silastic gastric banding (AGB).

In general, the gastric band is wrapped around an upper portion of the patient's stomach, forming a stoma that is less than the normal interior diameter of the stomach. This restricts food passing from an upper portion to a lower digestive portion of the stomach. When the stoma is of an appropriate size, food held in the upper portion of the stomach provides a feeling of fullness that discourages over eating.

As those skilled in the art will certainly appreciate, hybrid procedures involving gastric bypass and the utilization of a gastric band are becoming more and more common. These hybrid procedures involve placing a gastric band about the stomach in conjunction with the performance of the surgical procedure for gastric bypass surgery. However, significant challenges are associated with this procedure and, more specifically, some of these challenges relate to the placement of the gastric band directly over a staple line created as a result of the gastric bypass surgery. When a balloon type gastric band is positioned over a staple line, potential complications are encountered. These complications include damage to the gastric band resulting from the interaction of the gastric band with the staples in a manner potentially causing puncture of the gastric band by the staples. In addition to the potential puncture of the gastric band, the interaction of the gastric band with the staples may cause damage to the staple line due to rubbing of the staple line by positioning of the gastric band thereover and infection at the site of the gastric band placement.

### SUMMARY OF THE INVENTION

It is, therefore, an object of the present invention to provide an implantable restriction device including a belt and a balloon secured to the belt for engagement with tissue when the implantable restriction device is positioned about the stomach. A spacer is associated with the belt for maintaining the restriction device in a spaced relation with a staple line employed during gastric reduction surgery.

It is also an object of the present invention to provide an implantable restriction device wherein the spacer is formed from a bioabsorbable material.

It is another object of the present invention to provide an implantable restriction device wherein the bioabsorbable material has a predetermined degradation similar to the degradation of staples used in performing a medical procedure.

It is a further object of the present invention to provide an implantable restriction device wherein the spacer is composed of a shape memory material.

It is also an object of the present invention to provide an implantable restriction device wherein the spacer includes a first lateral side edge, a second lateral side edge, a top edge and a bottom edge, and the first lateral side edge is secured to the gastric band such that the long axis of the spacer is substantially aligned with the long axis of the gastric band.

It is another object of the present invention to provide an implantable restriction device wherein the distance between the first lateral side edge and the second lateral side edge substantially defines the circumferential extent of the spacer and is of sufficient length to cover the entire inner surface of the gastric band that will come into contact with a staple line, and the distance between the top edge and the bottom edge of the spacer defines the width of the gastric band the spacer will cover.

It is a further object of the present invention to provide an implantable restriction device wherein the spacer is integrally associated with the gastric band.

It is also an object of the present invention to provide an implantable restriction device wherein the spacer is attached along a lateral side of the gastric band, and the spacer includes a first side edge secured to the side edge of the belt of the gastric band and a second side edge which is free and extends away from the gastric band in the undeployed state of the spacer. With the first side edge of the spacer secured to the belt of the gastric band and the second side edge of the spacer extending away the spacer may be folded inwardly to a position covering the inner surface of the balloon when the gastric band is deployed about the stomach.

It is another object of the present invention to provide an implantable restriction device wherein two spacers laterally extend from the side edge of the belt of the gastric band.

It is a further object of the present invention to provide an implantable restriction device wherein the spacer is a buttress flap.

It is also an object of the present invention to provide an implantable restriction device wherein the gastric band includes a first end and a second end, and the buttress flap is secured to the second end of the gastric band and is oriented such that its longitudinal axis is perpendicular to the longitudinal axis of the gastric band when the gastric band is laid out on a flat surface. Thus, the buttress flap may be stapled to the sleeve portion created during the gastric reduction surgery when the gastric band is applied.

It is another object of the present invention to provide an implantable restriction device wherein the buttress flap is selectively secured to the gastric band.

It is a further object of the present invention to provide an implantable restriction device wherein the buttress flap includes an aperture through which the gastric band may pass.

It is also an object of the present invention to provide an implantable restriction device wherein the spacer includes a first buttress flap secured at a first end of the gastric band and a second buttress flap secured at a second end of the gastric band.

It is another object of the present invention to provide an implantable restriction device wherein the first buttress flap and the second buttress flap include mating coupling materials for selective engagement of the first buttress flap and the second buttress flap.

It is a further object of the present invention to provide an implantable restriction device wherein the mating coupling material is hook and loop coupling material.

It is also an object of the present invention to provide an implantable restriction device wherein the coupling material is a suture.

It is another object of the present invention to provide an implantable restriction device wherein the spacer is a clip extending between a first end and a second end of the belt.

It is a further object of the present invention to provide an implantable restriction device wherein the first end of the belt is provided with a hook member shaped and dimensioned for engagement with the clip and the second end of the belt is provided with a hook member shaped and dimensioned for engagement with the clip.

It is also an object of the present invention to provide an implantable restriction device wherein the clip secures the first end of the belt to the second end of the belt in facing relationship with tissue and a staple therebetween.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of a gastric band in accordance with a first embodiment being secured about a sleeve portion during a hybrid gastric procedure.

FIG. 2 is a perspective view of the gastric band shown in FIG. 1.

FIG. 3 is a top view of the gastric band shown in FIG. 2 with the gastric band secured about the sleeve portion.

FIGS. 4 and 5 are perspective views of a gastric band in accordance with an alternate embodiment with the spacer shown in its extended position and its use position.

FIGS. 6 and 7 are perspective views of a gastric band in accordance with an alternate embodiment with first and second spacers shown in their extended position and their use position.

FIG. 8 is a perspective view of an alternate gastric band in accordance with the present invention.

FIG. 9 is a top view of the gastric band shown in FIG. 8 with the gastric band secured about the sleeve portion.

FIG. 10 is a perspective view of an alternate gastric band in accordance with the present invention.

FIG. 11 is a top view of the gastric band shown in FIG. 10 with the gastric band secured about the sleeve portion.

FIG. 12 is a perspective view of an alternate gastric band in accordance with the present invention with the gastric band secured about the sleeve portion of a stomach.

FIG. 13 is a top view of the gastric band shown in FIG. 12 with the gastric band secured about the sleeve portion.

FIG. 14 is a perspective view of an alternate gastric band in accordance with the present invention with the gastric band secured about the sleeve portion of a stomach.

FIG. 15 is a top view of the gastric band shown in FIG. 14 with the gastric band secured about the sleeve portion.

FIG. 16 is a perspective view of an alternate gastric band in accordance with the present invention with the gastric band secured about the sleeve portion of a stomach.

FIG. 17 is a top view of the gastric band shown in FIG. 16 with the gastric band secured about the sleeve portion.

FIGS. 18, 19 and 20 are perspective views of the procedure for attaching an alternate gastric band about the stomach bridge during a gastric reduction procedure.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and for teaching one skilled in the art how to make and/or use the invention.

As discussed above, the present invention relates to an implantable restriction device. A preferred embodiment of the implantable restriction device is disclosed herein within reference to a gastric band used in restricting the effective size of the stomach for application in bariatric procedures. As such, the implantable restriction device of the present invention is referred to as a gastric band throughout the present disclosure, although those skilled in the art will appreciate the concepts underlying the present invention may be applied in a variety of implantable restriction devices.

The present invention provides for various gastric band constructions addressing the problems associated with hybrid gastric procedures combining surgical gastric reduction with gastric banding. The present invention, therefore allows for the placement of a gastric band 10 directly over a staple line 12 created in the formation of a sleeve portion 13 during the gastric bypass surgery of the hybrid procedure. The gastric band 10 constructions employed in accordance with the present invention protects the gastric band 10 from undesirable interaction with the staples by spacing the gastric band 10 from the staples in a manner minimizing the potential for puncture of the gastric band 10 by the staples and similar interactions which may cause damage to the staple line 12 due to rubbing of the staple line 12 by positioning of the gastric band 10 thereover. Infections at the placement site of the gastric band 10 are also minimized through utilization of the present gastric band 10.

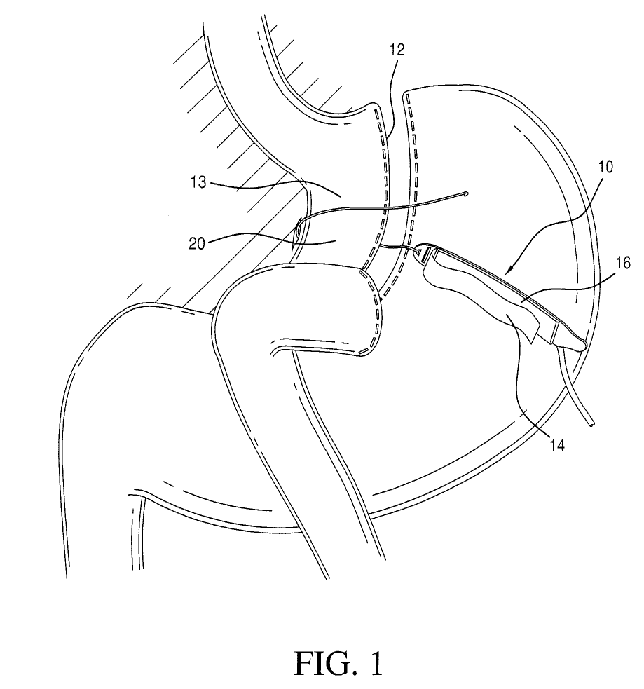

With reference to FIGS. 1, 2 and 3, and in accordance with a preferred embodiment of the present invention, a spacer 14 is secured along the internal surface 16 of the gastric band 10. Briefly, the spacer 14 is a flap secured at one end to the gastric band 10, in particular, the belt 30 of the gastric band 10. The spacer 14 is flexible and as such may be wrapped around the stomach. However, it also conforms to the inner surface 16 of the gastric band 10 such that upon application of the gastric band 10 it forms a smooth profile with both the gastric band 10 and the stomach 20 about which it is wrapped. In accordance with a preferred embodiment, the spacer 14 is preferably manufactured from plastic, rubber and/or other high durometer materials (when compared to the gastric band itself). In addition, it is contemplated the spacer 14 may be formed from a bioabsorbable material. By manufacturing the spacer 14 from a bioabsorbable material, the spacer 14 will slowly degrade over time just as the staple line 12 degrades over time. Specifically, once the staple line 12 heals and the staples no longer present a problem with regard to puncture of the balloon 32, it is no longer necessary to protect the inner surface 16 of the gastric band 10. Accordingly, by allowing the spacer 14 to degrade over time, the balloon 32 itself will directly come into contact with the stomach allowing for an improved, although thinner, interface between the stomach 20 and the gastric band 10. It is further contemplated the bioabsorbable material (for example, PDS or Vicrycl) may be manufactured in such a way that the degradation and strength of material during the cycle of degradation is predetermined and is selected so as to coincide with the degradation of the staples. Such a choice of materials would ensure that the protector is present until the staple line is healed.

Although it is contemplated various gastric band constructions may be employed in accordance with the present invention, a preferred gastric band 10 construction is disclosed. More details regarding gastric band constructions appropriate for use in conjunction with the present invention may be found in commonly owned U.S. patent application Ser. No. 11/798,634, entitled “GASTRIC BAND COMPOSED OF DIFFERENT HARDNESS MATERIALS”, which is incorporated herein by reference.

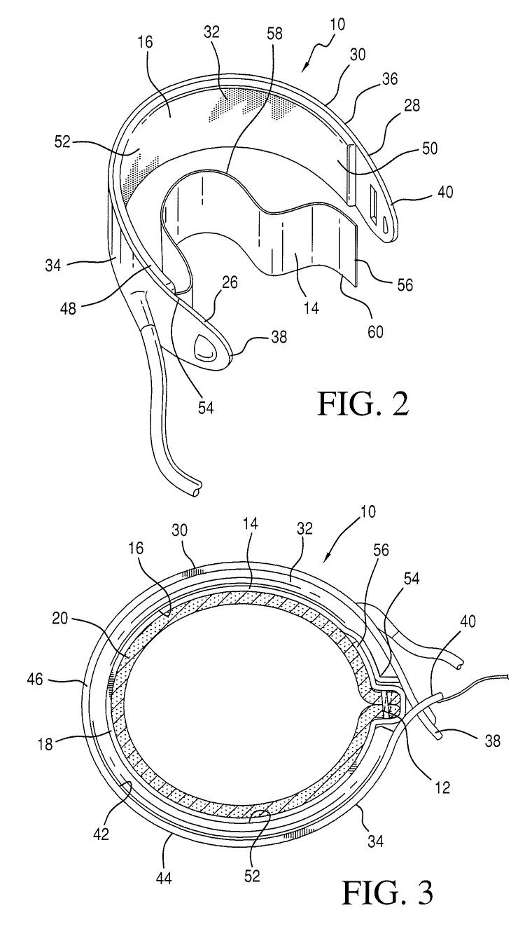

In general, and with reference to FIGS. 2 and 3, the gastric band 10 is a balloon-type gastric band and is generally composed of a reinforcing belt 30 to which an elongated balloon 32 is secured. The belt 30 includes a first end 34 and a second end 36 to which first and second latching members 38, 40 are respectively secured. The belt 30 further includes an inner surface 42 and an outer surface 44. The outer surface 44 is substantially smooth and forms a substantial portion of the outer surface of the gastric band 10 when it is secured about a patient's stomach. The inner surface 42 of the belt 30 is shaped and dimensioned for attachment to the outer surface 46 of the balloon 32.

With regard to the balloon 32, it also includes a first end 48, a second end 50, an inner surface 52 and an outer surface 46. The inner surface 52 is substantially smooth and is shaped and dimensioned for engaging the patient's stomach 20 when the gastric band 10 is secured thereto. The outer surface 46 of the balloon is shaped and dimensioned for coupling with the inner surface 42 of the belt 30.

The belt 30 and the balloon 32, and latching mechanism, that is, the first and second latching members 38, 40, are preferably manufactured from silicone. In accordance with a preferred embodiment, the gastric band 10 employs a flexible latching mechanism capable of locking and unlocking without destruction of the latching mechanism or significant reduction in retention capabilities after re-locking. The first and second latching members 38, 40 respectively act as both male and female members depending on the direction of motion and intent to lock or unlock the latching mechanism of the present gastric band.

Further details of the operation of the latching mechanism can be found in commonly owned U.S. patent application Ser. No. 11/182,072, entitled “LATCHING DEVICE FOR GASTRIC BAND”, which is incorporated herein by reference.

As briefly discussed above, the gastric band 10 is provided with a spacer 14 secured to the belt 30 for positioning along the inner surface 52 of the balloon 32 of the gastric band 10. The spacer 14 includes a first lateral side edge 54, a second lateral side edge 56, a top edge 58 and a bottom edge 60. The first lateral side edge 54 is secured to the belt 30 of the gastric band 10 such that the long axis of the spacer 14 is substantially aligned with the long axis of the gastric band 10. As will be appreciated based upon the following disclosure, the spacer 14 is flexible permitting it to extend across the inner surface 16 of the gastric band 10 in a manner creating a barrier between the balloon 32 of the gastric band 10 and the staples applied during the gastric bypass surgery by spacing the inner surface 52 of the balloon 32 from the staples of the staple line 12. The distance between the first lateral side edge 54 and the second lateral side edge 56 substantially defines the circumferential extent of the spacer 14 and is of sufficient length to cover the entire inner surface 16 of the gastric band 10 that will come into contact with the staple line 12. The distance between the top edge 58 and the bottom edge 60 of the spacer 14 defines the width of the gastric band 10 the spacer 14 will cover. The size of the spacer 14 should, therefore, be selected such that sufficient coverage is provided to ensure the staple line 12 will contact a portion of the spacer 14 regardless of the exact positioning of the gastric band 10 about the stomach.

In accordance with yet a further embodiment, a gastric band may be provided that covers the staple line to allow the gastric band to expand and shrink, but also protect the staples at all times. The staple line is also at risk when the gastric band is being positioned thereabout. The more precise or less traumatic the placement of the gastric band is, the less risk there is to the staple line itself. A guidewire attached to the band and to the tip of an introducer could help placing the band with less damage to the staple line. In accordance with this alternate embodiment, a protective cover is attached over the gastric band so as to protect it from the gastric bypass straps. The cover is resilient enough to minimize the potential for a staple puncturing the balloon of the gastric band but compliant and shaped appropriately to allow for wrapping around the new stomach pouch. As discussed above with the prior embodiments, the material may also be bioabsorbable. The bioabsorbable material may be manufactured as discussed above.

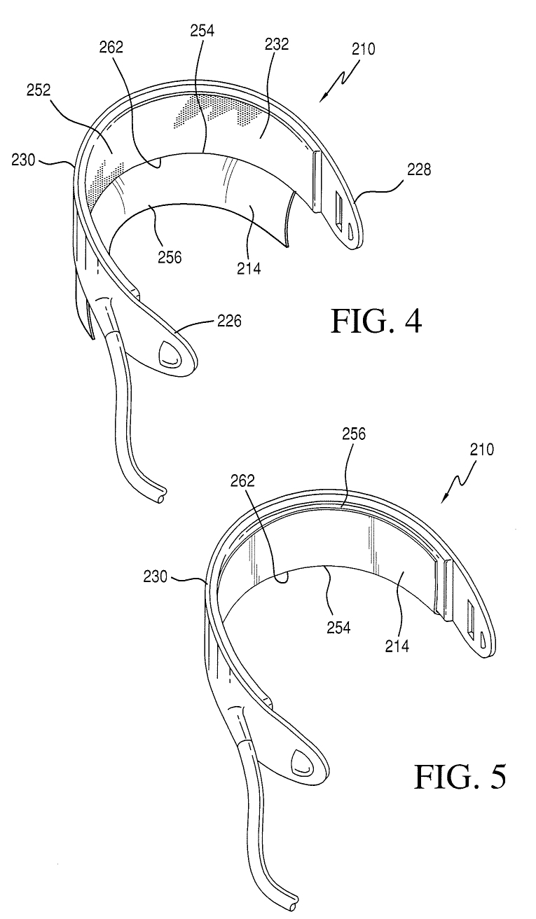

In accordance with yet a further embodiment and with reference to FIGS. 4 and 5, a spacer 214 is integrally associated with the gastric band 210. In particular, and in accordance with a first version of this embodiment, the spacer 214 is attached along a lateral side 262 of the belt 230 of the gastric band 210. The spacer 214 includes a first side edge 254 secured to the side edge 262 of the belt 230 of the gastric band 210 and a second side edge 256 which is free and extends away from the belt 230 of the gastric band 210 in the undeployed state of the spacer 214. The gastric band 210, in particular, the belt 230, further includes a first end 226 and a second end 228.

With the first side edge 254 of the spacer 214 secured to the belt 230 of the gastric band 210 and the second side edge 256 of the spacer 214 extending away, the spacer 214 may be folded inwardly to a position covering the inner surface 252 of the balloon 232 (that is, within the circumference defined by the gastric band 210) when the gastric band 210 is deployed about the stomach 20. The spacer 214 would be positioned such that it lies between the stomach tissue and the balloon 232 of the gastric band 210 upon deployment about the stomach. With this in mind, the spacer 214 is shaped and dimensioned to cover the full extent of the gastric band balloon 232.

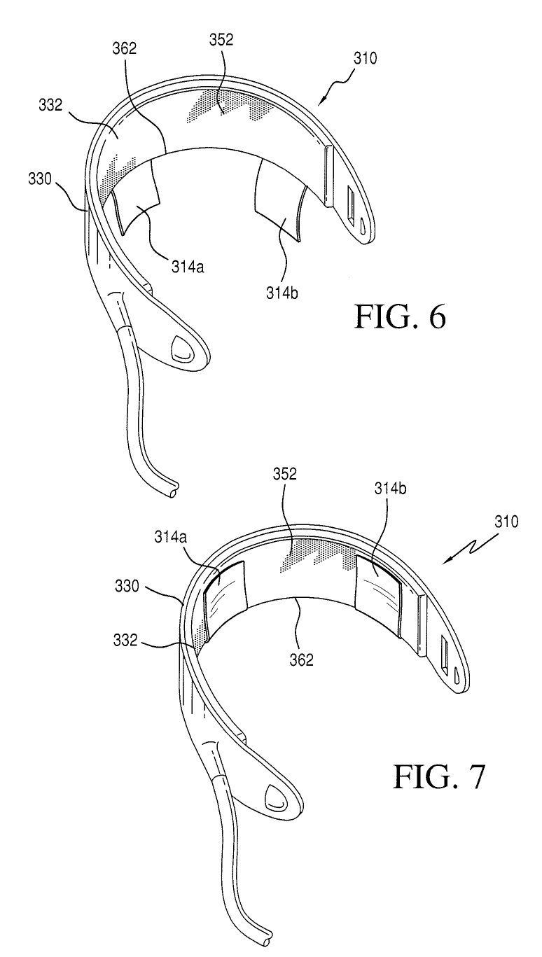

In accordance with an alternate embodiment, and with reference to FIGS. 6 and 7, the gastric band 310 is provided with two or more spacers 314a, 314b laterally extending from the side edge 362 of the belt 330 of the gastric band 310. These spacers 314a, 314b are positioned to provide an indication as to where the staple line 12 should be positioned along the gastric band 310; that is, between the spacers 314a, 314b when the gastric band 310 is open as shown in FIGS. 6 and 7. In addition, and upon folding the spacers 314a, 314b inwardly to a position adjacent the inner surface 352 of the balloon 332, the thickness of the spacers 314a, 314b spaces the inner surface 352 of the balloon 332 from the stomach 20 at the locations adjacent to the spacers 314a, 314b. By folding the spacers 314a, 314b inwardly in this manner upon deployment of the gastric band 310, pressure on the staple line 12 is reduced and the potential for damaging interaction between the staple line 12 and the inner surface 352 of the balloon 332 is reduced. The use of multiple spacers 314a, 314b also helps keep the gastric band 310 from buckling or folding in the area adjacent the staple line upon inflation thereof due to the thickness of the spacers 314a, 314b.

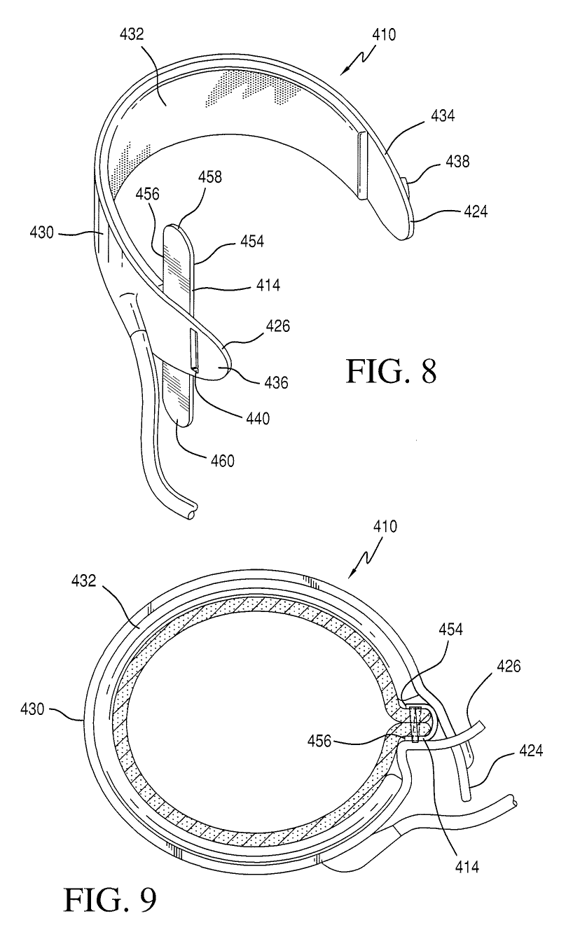

Referring now to FIGS. 8 and 9, an alternate embodiment of a gastric band 410 in accordance with the present invention is provided. The basic structure of the gastric band 410 is substantially the same as described above, although the protector is removed and the gastric band 410 is provided with a spacer in the form of an integrated buttress flap 414 to protect the balloon 432 of the gastric band 410 and the staples applied during the hybrid bypass procedure. In accordance with this embodiment, the balloon 432 and buttress flap 414 are integrated into a single product. As with the prior embodiment, this embodiment prevents the staples and the balloon 432 of the gastric band 410 from coming into contact. It is further contemplated this design may be incorporated into a new delivery device for applying the gastric band 410. In addition, this embodiment reduces procedure steps and prevents the gastric band 410 from migrating by attaching the buttress flap 414, staples and gastric band 410 above or below the balloon 432.

More particularly, and with reference to FIGS. 8 and 9, the gastric band 410 includes a first end 424 and a second end 426. The first end 424 includes a first latch member 438 (formed as part of the first end 434 of the belt 430) adapted for attachment to a second latch member 440 (also formed as part of the second end 436 of the belt 430) at the second end 426 of the gastric band 410. However, the second end 426, in addition to including the second latch member 440, includes a buttress flap 414 which is oriented such that its longitudinal axis is perpendicular to the longitudinal axis of the gastric band 410 when the gastric band 410 is laid out on a flat surface. As such, and upon application, the gastric band 410 is wrapped around the stomach and the buttress flap 414 is wrapped about the staple line. The buttress flap 414 is then stapled to the sleeve portion (above and/or below the balloon 432) created during the gastric reduction surgery. By stapling the buttress flap 414 to the sleeve portion, migration is prevented as discussed above

The buttress flap 414 includes a first lateral side edge 454, a second lateral side edge 456, a top edge 458 and a bottom edge 460. The first lateral side edge 454 is secured adjacent to the second latch member 440 at the second end 426 of the gastric band 410 such that the longitudinal axis extending from the top edge 458 of the buttress flap 414 to the bottom edge 460 of the buttress flap 414 is perpendicular to the longitudinal axis of the gastric band 410 as it extends from the first end 424 thereof to the second end 426 thereof (when the gastric band 410 is laid upon a flat surface). As with the prior embodiment, the buttress flap 414 is flexible permitting it to be wrapped about the staple line in a manner creating a barrier between the balloon 432 of the gastric band 410 and the staples applied during the gastric bypass surgery. The distance between the first lateral side edge 454 and the second lateral side edge 456 substantially defines the lateral extent of the buttress flap 414 and is of sufficient length to wrap about the staple line. The distance between the top edge 458 and the bottom edge 460 of the buttress flap 414 defines the length of the buttress flap 414 and should, therefore, be such that sufficient coverage is provided for the entire length of the staple line.

As with the prior embodiment, the buttress flap 414 is preferably manufactured from plastic, rubber and/or other high durometer materials (when compared to the gastric band itself). In addition, it is contemplated the buttress flap 414 may be formed from a bioabsorbable material as discussed above.

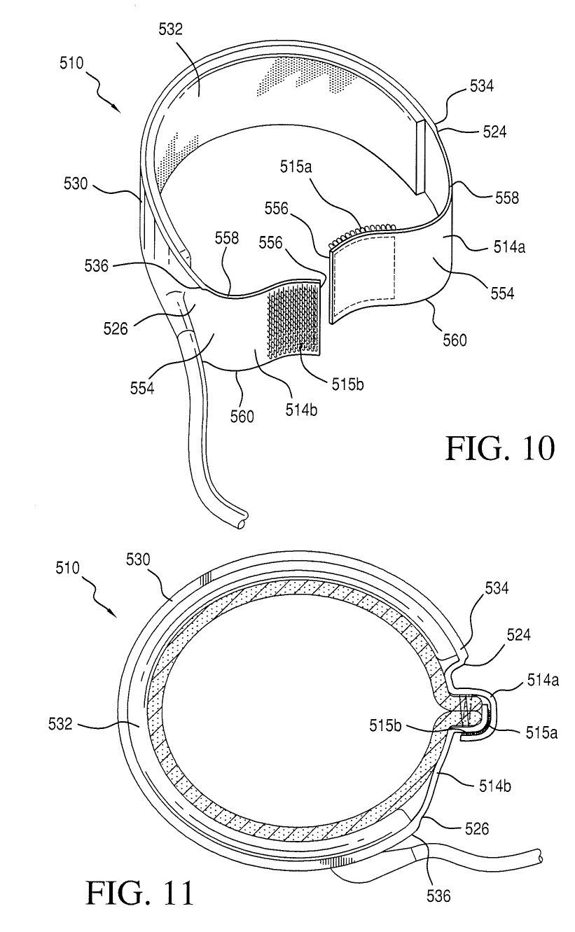

In accordance with yet another embodiment and with reference to FIGS. 10-11, the balloon 532 is formed with first and second spacers in the form of first and second buttress flaps 514a, 514b respectively secured to the gastric band 510 adjacent the first and second ends 524, 526 of the gastric band 510, and, in particular, to the first and second ends 534, 536 of the belt 530. The first and second buttress flaps 514a, 514b respectively include hook and loop type material 515a, 515b allowing for selectively coupling of the first and second buttress flaps 514a, 514b in a manner discussed below in greater detail. As a result, and in contrast to the previous fastening mechanism, the hook and loop material is utilized to secure the first and second ends 524, 526 of the gastric band 510 at a position adjacent the staple line 512 formed in accordance with the gastric reduction procedure, thus obviating the need for the latching members discussed above. By positioning the first and second buttress flaps 514a, 514b at a location in alignment with the staple line 512, the balloon 532 of the gastric band 510 will not come into contact with the staple line and thereby avoids unnecessary contact with the staple line. In addition, by forming the first and second buttress flaps 514a, 514b such that the hook and loop material engage each other along the inner surface of the gastric band 510 and providing a smooth outer surface for the first and second buttress flaps 514a, 514b, the smooth side of the buttress flaps 514a, 514b contacts tissue and obviates any undesirable contact therebetween.

More particularly, each of the first and second buttress flaps 514a, 514b includes a first lateral side edge 554, a second lateral side edge 556, a top edge 558 and a bottom edge 560. The first lateral side edge 554 is secured adjacent to the respective first and second ends 534, 536 of the belt 530 of the gastric band 510 such that the longitudinal axis extending from the top edge 558 of the buttress flaps 514a, 514b to the bottom edge 560 of the buttress flaps 514a, 514b is perpendicular to the longitudinal axis of the gastric band 510 as it extends from the first end 524 thereof to the second end 526 thereof (when the gastric band 510 is laid upon a flat surface). The second lateral side edge 556 of the buttress flap 514a is provided with a hook or loop type material shaped and dimensioned to engage to the hook or loop type material positioned at the second lateral side edge 556 of the other buttress flap 514b.

As with the prior embodiment, the buttress flaps 514a, 514b are flexible permitting them to be wrapped about the staple line with second lateral side edges 554 of the respective first and second buttress flaps 514a, 514b secured via the hook and loop type material in a manner creating a barrier between the balloon 532 of the gastric band 510 and the staples applied during the gastric bypass surgery. The combined distance between the second lateral side edges 554 of the first and second buttress flaps 514a, 514b substantially defines the lateral extent of the first and second buttress flaps 514a, 514b as they wrap about the staple line and is of sufficient length to wrap about the staple line when the first and second buttress flaps 514a, 514b are secured to each other. The distance between the top edge 558 and the bottom edge 560 of the respective first and second buttress flaps 514a, 514b defines the length of the buttress flap 514a, 514b and should, therefore, be such that sufficient coverage is provided for the entire length of the staple line.

As with the prior embodiment, the first and second buttress flaps 514a, 514b may be manufactured from plastic, rubber and/or other high durometer materials (when compared to the gastric band itself). In addition, it is contemplated the first and second buttress flaps 514a, 514b from a bioabsorbable material as discussed above.

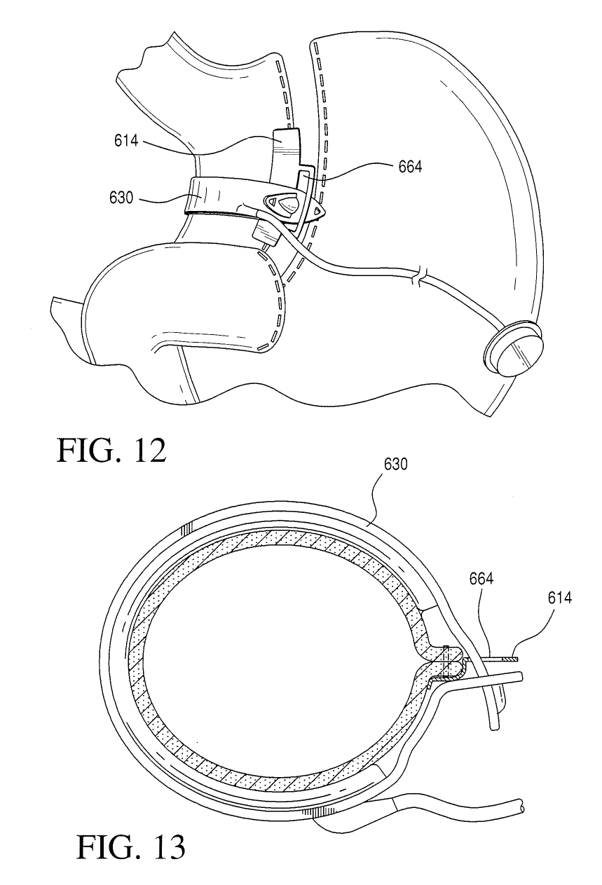

Referring now to FIGS. 12 and 13, the buttress flap 614, regardless of the embodiment employed in their manufacture, may be formed separately from the gastric band. In accordance with such an embodiment, the buttress flap 614 is constructed in much the same manner as discussed in accordance with the various embodiments disclosed herein, but includes an aperture 664 for receiving the gastric band 610, in particular, the belt 630, in a manner retaining the buttress flap 614 in position covering the staple line.

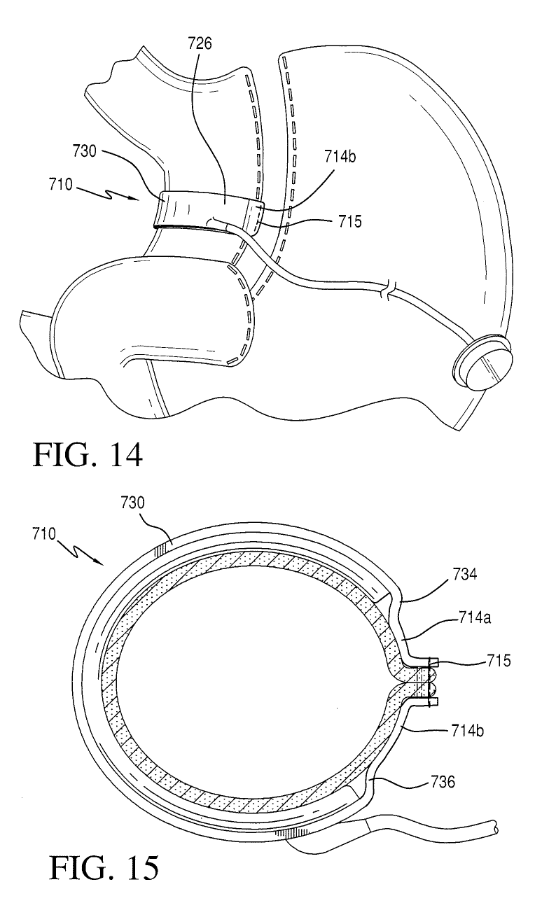

In accordance with an alternate embodiment, and with reference to FIGS. 14 and 15, the gastric band 710 is similarly formed with first and second buttress flaps 714a, 714b at respective first and second ends 734, 736 of the belt 730 of the gastric band 710. However, and in contrast to the embodiment described above, the first and second buttress flaps 714a, 714b, and ultimately, the first and second ends 734, 736 of the belt 730 of the gastric band 710, are connected by a suture line 715 alternately threaded through the first and second buttress flaps 714a, 714b. The suture line is 715 threaded such that the first and second buttress flaps 714a, 714b may be drawn toward each other in a manner securing both the first and second buttress flaps 714a, 714b and the gastric band 710 to the stomach. As with the embodiment disclosed above with reference to FIGS. 8 and 9, the buttress flaps may be secured to the sleeve by staples so as to assist in preventing migration of the buttress flaps and the gastric band.

Various embodiments employing buttress flaps are disclosed above. It is contemplated that these embodiments may be manufactured with buttress flaps composed of a shape memory material (or other spring like materials) facilitating the positioning and application of the buttress flaps. For example, the buttress flaps, upon deployment within the body, would reshape (for example, from a flat predeployment orientation) into a desired coil orientation for covering the staple line.

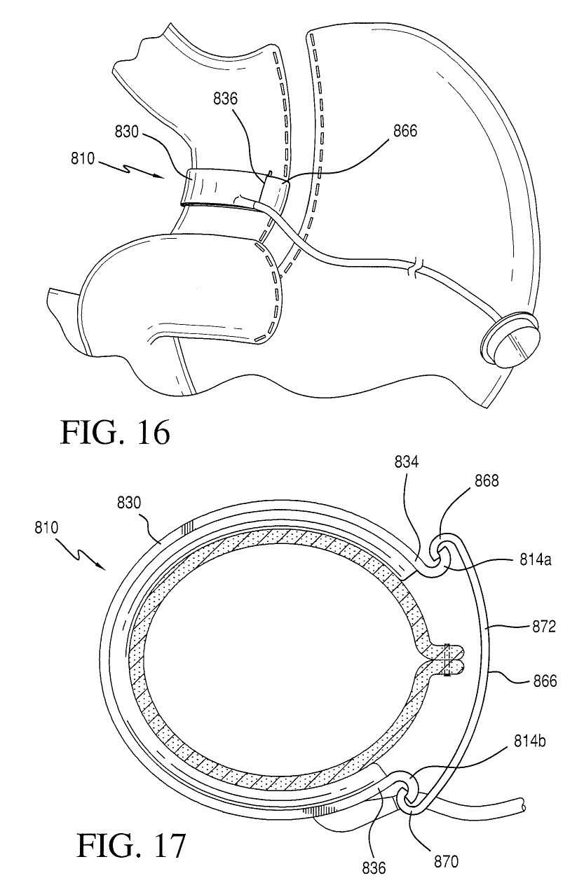

Similarly, and with reference to FIGS. 16 and 17, the first and second ends 834, 836 of the belt 830 of the gastric band 810 are secured together via a plastic clip or metal string 866. This concept is also designed to assist in addressing challenges associated with the placement of the gastric band 810 directly over a staple line so as to reduce the potential for complications and damage to the gastric band 810, damage to the staple line and infection at the site of the band placement. In accordance with this embodiment, a plastic clip/metal string 866 is secured to the respective first and second hook members 814a, 814b at the first and second ends 834, 836 of the belt 830 of the gastric band 810 for securing the passage of the gastric band 810 about the stomach. The plastic clip/metal string 866 is used where the gastric band 810 would be in contact with the staple line thereby reducing the chance of damage to the gastric band 810. In accordance with the embodiment disclosed herein, the component is substantially circular in configuration and includes a first end 868 secured to the first hook member 814a and a second end 870 secured to the second hook member 814b. Connecting the first end 868 of the clip 866 and the second end 870 of the clip 866 is a circular shape member 872. As such, when the first hook member 814a and the second hook member 814b are drawn together based upon the inherent bias of the clip 866, the circular configuration of the plastic clip 866 results in the clip 866 being spaced from the staple line.

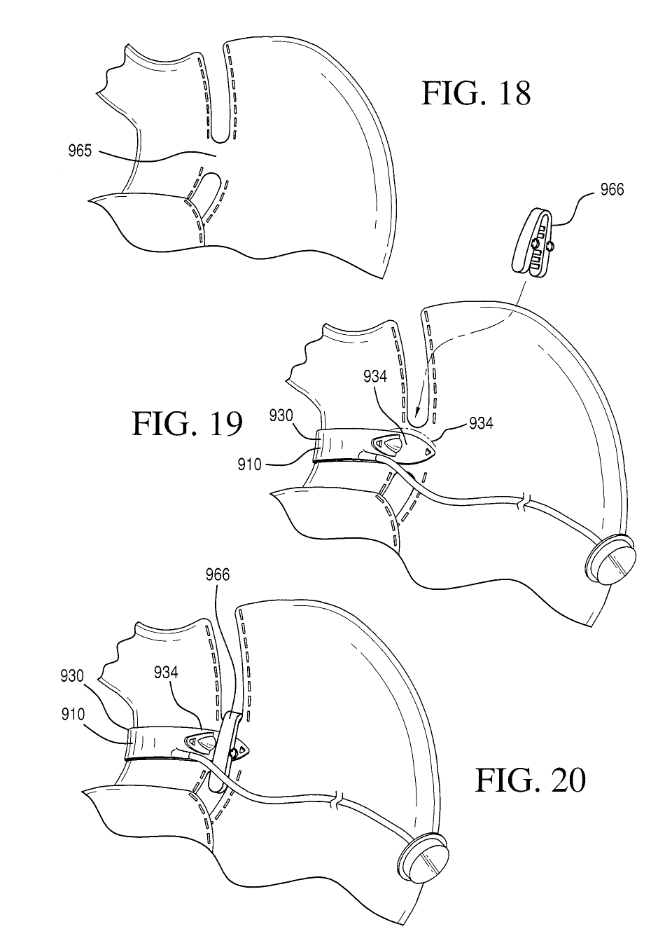

In accordance with yet another embodiment, and with reference to FIGS. 18, 19 and 20, the gastric band 910 is provided with a clip 966 that locks around the stomach bridge 965. The stomach bridge is the unstapled open path from the small pouch to the large part of the stomach, note 965 in FIG. 18. The clip 966 works as a dam preventing passage of food therethrough. Instead of the gastric band using fastening members at its first and second ends, the clip 966 fastens the ends of the gastric band 910 such that the first end 934 of the belt 930 and the second end 936 of the belt 930 are in facing relationship with the stomach bridge therebetween while also preventing the passage of food through the bridge 965 as best seen in FIG. 20.

Although the present invention is described for use in conjunction with gastric bands, those skilled in the art will appreciate the above invention has equal applicability to other types of implantable bands, for example, bands that are used for the treatment of fecal incontinence. One such band is described in U.S. Pat. No. 6,461,292. Bands can also be used to treat urinary incontinence. One such band is described in U.S. Patent Application Publication No. 2003/0105385. Bands can also be used to treat heartburn and/or acid reflux. One such band is described in U.S. Pat. No. 6,470,892. Bands can also be used to treat impotence. One such band is described in U.S. Patent Application Publication No. 2003/0114729.

While the preferred embodiments have been shown and described, it will be understood that there is no intent to limit the invention by such disclosure, but rather, is intended to cover all modifications and alternate constructions falling within the spirit and scope of the invention.

1. An implantable restriction device, comprising:
a belt;
a balloon secured to the belt for engagement with tissue when the implantable restriction device is positioned about the stomach; and
a spacer associated with the belt for maintaining the restriction device in a spaced relation with a staple line employed during gastric reduction surgery.

2. The implantable restriction device according to claim 1, wherein the spacer is formed from a bioabsorbable material.

3. The implantable restriction device according to claim 2, wherein the bioabsorbable material has a predetermined degradation similar to the degradation of staples used in performing a medical procedure.

4. The implantable restriction device according to claim 1, wherein the spacer is composed of a shape memory material.

5. The implantable restriction device according to claim 1, wherein the spacer includes a first lateral side edge, a second lateral side edge, a top edge and a bottom edge, and the first lateral side edge is secured to the gastric band such that the long axis of the spacer is substantially aligned with the long axis of the gastric band.

6. The implantable restriction device according to claim 5, wherein a distance between the first lateral side edge and the second lateral side edge substantially defines the circumferential extent of the spacer and is of sufficient length to cover the entire inner surface of the gastric band that will come into contact with a staple line, and the distance between the top edge and the bottom edge of the spacer defines the width of the gastric band the spacer will cover.

7. The implantable restriction device according to claim 1, wherein the spacer is integrally associated with the gastric band.

8. The implantable restriction device according to claim 7, wherein the spacer is attached along a lateral side of the gastric band, and the spacer includes a first side edge secured to the side edge of the belt of the gastric band and a second side edge which is free and extends away from the gastric band in the undeployed state of the spacer, wherein with the first side edge of the spacer secured to the belt of the gastric band and the second side edge of the spacer extending away, the spacer may be folded inwardly to a position covering the inner surface of the balloon when the gastric band is deployed about the stomach.

9. The implantable restriction device according to claim 1, wherein two spacers laterally extend from the side edge of the belt of the gastric band.

10. The implantable restriction device according to claim 1, wherein the spacer is a buttress flap.

11. The implantable restriction device according to claim 9, wherein the gastric band includes a first end and a second end, and the buttress flap is secured to the second end of the gastric band and is oriented such that its longitudinal axis is perpendicular to the longitudinal axis of the gastric band when the gastric band is laid out on a flat surface, thus the buttress flap may be stapled to the sleeve portion created during the gastric reduction surgery when the gastric band is applied.

12. The implantable restriction device according to claim 10, wherein the buttress flap is selectively secured to the gastric band.

13. The implantable restriction device according to claim 12, wherein the buttress flap includes an aperture through which the gastric band may pass.

14. The implantable restriction device according to claim 1, wherein the spacer includes a first buttress flap secured at a first end of the gastric band and a second buttress flap secured at a second end of the gastric band.

15. The implantable restriction device according to claim 14, wherein the first buttress flap and the second buttress flap include mating coupling materials for selective engagement of the first buttress flap and the second buttress flap.

16. The implantable restriction device according to claim 15, wherein the mating coupling material is hook and loop coupling material.

17. The implantable restriction device according to claim 15, wherein the coupling material is a suture.

18. The implantable restriction device according to claim 1, wherein the spacer is a clip extending between a first end and a second end of the belt.

19. The implantable restriction device according to claim 18, wherein the first end of the belt is provided with a hook member shaped and dimensioned for engagement with the clip and the second end of the belt is provided with a hook member shaped and dimensioned for engagement with the clip.

20. The implantable restriction device according to claim 18, wherein the clip secures the first end of the belt to the second end of the belt in facing relationship with tissue and a staple therebetween.
